(19) United States
(12) Patent Application Publication
(10) Pub. No.: US 2007/0260225 A1
(43) Pub. Date: Nov. 8, 2007

(54) STEERABLE SHEATH ACTUATOR

(75) Inventors: Ghassan Sakakine, Rancho Santa Margarita, CA (US); Haruyasu Yawata, Huntington Beach, CA (US)

Correspondence Address: APPLIED MEDICAL RESOURCES CORPORATION, 22872 Avenida Empresa, Rancho Santa Margarita, CA, 92688, US

(73) Assignee: APPLIED MEDICAL RESOURCES CORPORATION, Rancho Santa Margarita, CA (US)

(21) Appl. No.: 11/750,235

(22) Filed: May 17, 2007

Related U.S. Application Data
(63) Continuation-in-part of application No. 11/152,945, filed on Jun. 14, 2005.
Application No. 11/152,945 is a continuation-in-part of application No. 10/832,867, filed on Apr. 26, 2004.
Application No. 11/152,945 is a continuation-in-part of application No. 10/766,138, filed on Jan. 28, 2004.
Application No. 11/152,945 is a continuation-in-part of application No. 10/298,116, filed on Nov. 15, 2002, now Pat. No. 7,005,026.
(60) Provisional application No. 60/747,512, filed on May 17, 2006.
(60) Provisional application No. 60/579,500, filed on Jun. 14, 2004.

(51) Int. Cl. A61M 25/08

(52) U.S. Cl. 604/528

(57) ABSTRACT

Provided is an actuator for a steerable sheath that provides some or all of the following features: operability with one hand, ergonomic and comfortable to use, accuracy during sheath tip positioning, positive locking mechanism to maintain sheath tip position, and a fastening device to secure the actuator to a surgical drape or other object

### CROSS-REFERENCE TO RELATED APPLICATION

This application claims benefit of U.S. Provisional Application No. 60/747,512, filed May 17, 2006, and is a continuation-in-part of U.S. patent application Ser. No. 11/152,945, filed Jun. 14, 2005, the entire disclosure of which is hereby incorporated by reference as if set forth in full herein. U.S. patent application Ser. No. 11/152,945 is a continuation-in-part of U.S. patent application Ser. No. 10/832,867, filed Apr. 26, 2004, Ser. No. 10/766,138, filed Jan. 28, 2004 and Ser. No. 10/298,116, filed Nov. 15, 2002, and claims benefit of U.S. Provisional Application No. 60/579,500, filed Jun. 14, 2004, the entire disclosures of which are hereby incorporated by reference as if set forth in full herein.

### BACKGROUND

The present invention generally relates to surgical access devices and, more specifically, to actuators that provide for steering in sheaths that are useful in vascular procedures.

Sheaths and catheters have long been used to access body conduits such as the arterial and venous branches of the vascular system, urinary tract, body cavities such as the thorax and abdomen, and hollow viscous organs such as the stomach, intestines and urinary bladder. More specifically, sheaths and catheters have been used for fluid delivery, fluid recovery, implant delivery and for providing an access pathway for an instrument such as an endoscope. However, many endoscopes, for example, are flexible enough to bend but are not steerable or deflectable in a controlled and/or dynamic manner As such, there is a desire in the art for a steerable access sheath that is able to perform intricate manipulations through vessels, body cavities and/or tissue For some instruments, steering has been achieved, for example, by “pre-bending” the distal tip of a surgical device before insertion and then rotating the device once it has been inserted and has reached a branch artery inside the body. If the angle of the bend has to be adjusted, then the device may have to be removed, re-bent and reinserted. This may result in greater time spent in the body and thereby increase surgery time. Furthermore, since these sheaths and catheters navigate many hard-to-reach areas, it may be desirable that these devices be stiff and yet as flexible as possible. It may also useful that the sheaths and catheters are constructed with thin walls to minimize the diameter of the device and to maximize the radii of the internal lumen.

If the access sheath is constructed with a thin wall made of a plastic or rubber material, the sheath may bend or twist during use. This may result in potential damage as the sharp edge of the kinked sheath may allow an endoscope or other device to complicate the surgical procedure. Moreover, a bent or kinked sheath may be useless because it cannot communicate and it may not allow the passage of an instrument. As such, there is a desire in the art for a steerable access sheath that is durable enough to provide sufficient strength and stiffness to be guided through a body cavity or tissue and, at the same time, be flexible enough to perform intricate manipulations through the body cavity or tissue.

In providing steering to the access sheath, it is desirable to provide a handle or actuator that can assist in accurately articulating the sheath tip remotely and in maintaining the tip in the desired position for as long as required by the operator. It is also desirable to provide comfort and ease of use to the operator, and to provide a means for securing the sheath and/or sheath actuator so as to prevent unintended disruption or movement of the sheath. What is needed, therefore, is an actuator for a steerable sheath that provides some or all of the following features: operable with one hand, ergonomic and comfortable to use, accuracy during sheath tip positioning, positive locking mechanism to maintain sheath tip position, and a fastening device to secure the actuator to a surgical drape or other object

### SUMMARY

In one embodiment, the present invention is directed to a handle or actuator for use with a steerable surgical device, the handle comprising a housing, an axle disposed within the housing, a plurality of teeth radially disposed around the axle, a movable knob connected to and outside the housing, the axle connected to the knob such that movement of the knob in a first direction causes the axle to rotate clockwise and movement of the knob in a second direction causes the axle to rotate counterclockwise, at least one hard-stop on the housing, the hard-stop adapted to limit movement of the knob and/or rotation of the axle, a loaded spring disposed within the housing, the loading spring having a cantilever arm adapted to engage the plurality of teeth around the axle and a movable button extending through an opening in the housing, such that movement of the button in a first direction causes the cantilever arm to engage the plurality of teeth and movement of the button in a second direction causes the cantilever arm to disengage from the plurality of teeth.

In another embodiment, the present invention is directed to a surgical access device comprising a tube having a substantially rigid portion and a substantially flexible portion extending from the substantially rigid portion, the tube including a primary lumen and a secondary lumen both extending through the tube, the secondary lumen having a pull wire extending through the secondary lumen and connected to the flexible portion of the elongated body, a connector having a distal end connected to the tube, a proximal end including a funnel-shaped portion, and a channel disposed between the distal end and the proximal end, the pull wire extending through the connector from the distal end through the channel, a flexible tubing connected to the channel through which the pull wire extends, and a handle connected to the flexible tubing and including a housing, an axle disposed within the housing and connected to the pull wire, a plurality of teeth radially disposed around the axle, a movable knob connected to and outside the housing, the axle connected to the knob, and a loaded spring disposed within the housing, the loading spring having a cantilever arm adapted to engage the plurality of teeth around the axle and a movable button extending through an opening in the housing.

Optionally, the handle and/or surgical access device may also include one or more directional indicators to show sheath position, means for locking deflection of the sheath, means for providing incremental deflection of the sheath, means for preventing over-tensioning of the pull wire, and/or a clip for securing the handle and/or surgical access device to a surgical drape.

### BRIEF DESCRIPTION OF THE DRAWINGS

FIG. 1 illustrates a surgical access device or steerable access sheath in accordance with one aspect of the invention;

FIG. 2 is a cross sectional view of the access device of FIG. 1;

FIG. . 3 illustrates a dilator in accordance with one aspect of the present invention;

FIG. 4 illustrates a surgical access device or steerable access sheath in accordance with one aspect of the present invention;

FIG. 5 illustrates a surgical access device or steerable access sheath in accordance with another aspect of the present invention;

FIG. 6 illustrates a surgical access device or steerable kink resistant access device in accordance with one embodiment of the present invention;

FIG. . 7 is a front view of the distal end of the access device of FIG. 6;

FIG. 8 is a rear view of the proximal end of the access device of FIG. 6;

FIG. 9 is an enlarged side view of the distal portion of the access sheath of FIG. . 6;

FIG. 10 is a side-section view of the distal portion of the access sheath of FIG. 9;

FIG. 11 illustrates a steerable kink resistant access device of the present invention with its distal portion deflected;

FIG. 12 is a top view of the distal portion of the access sheath of the present invention;

FIG. 13 is a bottom view of the distal portion of the access sheath of the present invention;

FIG. 14 illustrates the atraumatic distal end of the access sheath of the present invention;

FIG. 15 illustrates an actuator of the access device of the present invention used to control the steerable region or portion of the access sheath;

FIG. 16 illustrates the access device of the present invention guiding a scope into a kidney pole;

FIG. 17 illustrates a perspective view of the distal portion of an access sheath having a flattened tensioning member;

FIG. 18 illustrates a side-elevation view illustrating a spring embodiment of the tube associated with the sheath of the present invention;

FIGS. 19-22 illustrate cross-sectional views of embodiments of an access sheath in various stages of fabrication in accordance with the present invention;

FIG. 23 illustrates a perspective view of an actuation hand-piece in accordance with one embodiment of the invention;

FIG. 24 illustrates a cross-sectional view of the actuation hand-piece of FIG. 23;

FIG. 25 illustrates a perspective view of a disassembled actuation hand-piece of FIG. 23;

FIG. 26 illustrates a (a) top, (b) side and (c) bottom view of the actuation hand-piece of FIG. 23;

FIG. 27 illustrates the cantilever arm of the actuation hand-piece of FIG. 23 in an (a) on and (b) off position; and

FIG. 28 illustrates a cross-sectional view of a connector in accordance with one embodiment of the present invention.

### DETAILED DESCRIPTION

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the structures and/or methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

Many of the attendant features of this invention will be more readily appreciated as the same becomes better understood by reference to the following detailed description and considered in connection with the accompanying drawings in which like reference symbols designate like parts throughout

Steerable Access Sheath

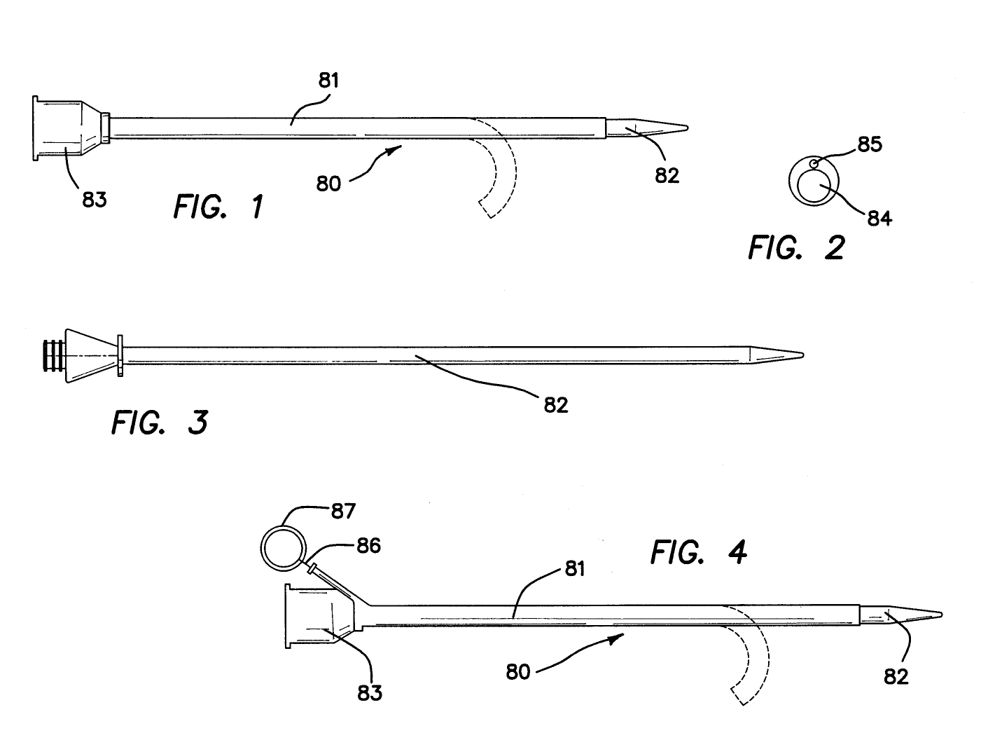

In FIGS. 1-4, the steerable access sheath 80 includes an elongate body 81 and, in one embodiment, a funnel or tapered entry 83. The elongate body 81 is substantially or completely steerable and may have a variable stiffness or flexibility or is fully pliable. The outside diameter of the elongate body is also sufficiently small so that it may be inserted into a reduced or minimally sized body cavity or conduit, e.g., a vein or artery. The access sheath 80 further includes a primary lumen 84 and a secondary lumen 85 both extending through the elongate body 81. The primary lumen 84 is sized and configured to provide an access pathway to a surgical site or a target site for the surgical procedure. For example, primary lumen 84 provides a conduit to advance a surgical instrument, e.g., a dilator, or diagnostic and therapeutic elements, e.g., a contrast agent, to the surgical or target site. The secondary lumen 85 is sized and configured to contain a tensioning device 86 such as a control or pull wire that, when acted upon, will deflect the elongate body 81 of the access sheath 80. In one embodiment, a secondary lumen is not utilized and thus the tensioning device 86 is directly included with the access sheath 80. For example, the tensioning device 86 may be secured to the access sheath or primary lumen and extend along the length of the access sheath or primary lumen to provide sufficient deflection of the elongate body 81 of the access sheath 80 via the tensioning device 86. In one aspect, tensioning device 86 may be embedded in the wall of the access sheath 80 and/or the primary lumen 84.

The tensioning device 86 extends through the secondary lumen 85 and is attached to an actuator 87 at one end and to a distal portion of the elongate body 81 at the other end. The actuator 87 may include a thumb-actuated knob, a ring, as illustrated, or another type of device to control the tensioning device 86. As shown, a ring connected to a pull wire may be drawn proximally to provide tension to the tensioning device 86. When the ring is released, the pull wire moves distally to loosen tension or cause the tensioning device 86 to loosen to allow the access sheath to straighten or return back to a previous or initial form.

As such, by manipulating the actuator 87, a user can steer the access sheath 80 to navigate circuitous or torturous conduits or cavities within the body to access the surgical site or point of interest. Additionally, the access sheath via the primary lumen provides a conduit or a channel from outside the body to the point of interest for the insertion and withdrawal of instruments, tissue or other items used for or in conjunction with the surgical procedure.

It is appreciated that the actuator 87 may resemble, emulate, embody or otherwise incorporate the actuation hand-pieces described in previous or the following embodiments and may be in-line, offset or remote from the access sheath. Additionally, the access sheath may comprise a plurality of pull wires attached to a plurality of thumbwheels, axles, knobs or other types of movable components of an actuator or actuation hand-piece to deflect the access sheath in one or more different directions.

In one particular embodiment, the funnel-shaped entry 83 is sized and configured to guide a dilator, an obturator and/or other instrumentation into a working channel to form a transition into the primary lumen of the access sheath 80. The funnel-shaped entry also includes or is connected to a connector to provide a conduit that connects the secondary lumen and tensioning device 86 to the actuator 87. In one embodiment, the entry 83 includes or is connected to a valve, such as a zero and/or septum valve, which is fixed or floats. The valve may also include deformable material, construction, gel or any combination thereof to form a seal around instruments and the like inserted in to the entry or to seal the entry after or prior to instruments and the like being removed or inserted in the entry 83.

The access sheath 80 and various embodiments of access sheaths and actuators or actuation hand-pieces previously described, here now referred to as the access sheath, in accordance with one aspect of the invention is applicable in vascular procedures and in other procedures among other fields, such as cardiology, urology, radiology, electrophysiology and gastroenterology. For example, in interventional radiology or interventional nephrology in which guided imaging is utilized, the access sheath 80 being steerable and appropriately sized assists in the placement of instruments, solutions or agents used in these procedures.

In one embodiment, the access sheath is combined with an instrument or device used to stretch or enlarge an opening, e.g., a dilator, which allows for gradual and atraumatic dilation of the artery or vein while the access sheath is being placed. Once the access sheath has been placed at a desired location, the dilator is removed and the access sheath is left in place. The access sheath allows for continued access to the desired area, for example, for the placement of surgical and/or therapeutic instruments or agents, while providing protection of the vessel. Continuous access provided by the access sheath may also reduce the need to re-locate a site or vessel. Additionally, with the access sheath being deflectable or steerable, the user may effectively and efficiently navigate the intricate and sometimes extensive circulatory system. As such, placement of instruments through the primary lumen of the access sheath at or proximal the operation site can be achieved by dynamically steering and/or continuously steering the access sheath.

In another embodiment, the access sheath being steerable provides direct and proximal vascular access to circulatory vessels or specific organ or tissue to ensure that healthy blood flow or the ability to deliver therapeutic agents is maintained. For example, for hemodialysis, regular vascular access to circulatory system can be provided by the access sheath or for cancer, chemotherapy via vascular access to the circulatory system can be provided by the access sheath.

The access sheath may also be useful in diagnostic radiography, which confirms the presence of an occlusion of vessels, e.g., lesion or thrombus formations. When performed at or near junction of an artery and a vein, such as a fistula, directing the contrast agent utilized such that the agent flows towards the downstream vessels of interest may be difficult. By deflecting or changing the shape of the access sheath to conform to the shape of the vessel(s), the access sheath allows the contrast agent to be directed in the antegrade flow of the artery and/or vein.

In one aspect, an access sheath 80 with a long length, e.g., over 100 centimeters, may also be useful for specific surgical, therapeutic or diagnostic procedures for various diseases or conditions, e.g., embolization and in particular, uterine fibroid embolization. The access sheath being able to have a long length does not restrict the path taken to reach the tissue, vessel or area of interest. The steerable access sheath 80 also eases the navigation of the circuitous path from the femoral arteries, the iliac arteries to the uterine arteries. For example, by deflecting the access sheath 80 making the turn, bend or course change from one artery to another, e.g., from the iliac to the femoral artery or from the femoral artery to the uterine artery, is made easier. With the access sheath placed at or near the area of interest, the primary lumen provides the conduit for the insertion of agents, e.g., biocompatible occlusion particles, or other treatment or diagnostic agents, solutions or devices.

The primary lumen of the access sheath 80 can also provide a fixed size to accommodate or overcome limitations imposed by the length of the surgical instrument to be inserted, the size of a vessel relative to the instrument and/or the blood flow around the instrument. In one embodiment, the distal end of the access sheath is tapered and thus has a smaller diameter than the proximal end of the access sheath. The primary lumen and secondary lumen diameters, however, remain substantially constant throughout the access sheath.

In one embodiment, one or more coated wires are wound around the inner/outer periphery of the access sheath, the primary lumen and/or any combination thereof to strengthen the access sheath, such that a flexible, pre-bendable or otherwise not actively controllable instrument may be controllably deflected dynamically as the access sheath is controlled. Additionally, an actively deflectable surgical instrument may have a complicated construction providing components, e.g., optics or clamps, to perform its surgical function and components to perform the active deflection. Therefore, such instruments may be fragile or if broken may be expensive to replace or repair or still usable as a surgical instrument but not actively deflectable. Also, the vessel or body conduit accessed or the surgical procedure performed may impose size limitations to prevent the inclusive of deflectable components or mechanisms in the surgical instruments.

As such, the access sheath may replace the components or use of the components in such surgical instruments or induce a broken instrument to be controllably deflected thereby reducing replacement, repair and/or construction costs, reducing wear and tear of such instruments and increasing the life of such instruments. Also, the reinforced access sheath allows the size and shape of the primary lumen to remain substantially constant throughout the access sheath, thereby reducing forces on instruments placed within the access sheath, which may extend the life of these instruments.

The forces or stress accumulated along the access sheath that may cause kinks in the access sheath are also distributed along the access sheath due to the composite construction of the access sheath. Thus, kinks in the access sheath are reduced. The wire coil(s) may also allow the access sheath walls to be very thin without reducing durability or strength in the access sheath. Thus, the overall or outer diameter of the access sheath may be small, which may also reduce the incision or insertion point for the access sheath, without reducing the size or diameter of the primary lumen.

As such, the access sheath of various embodiments of the present invention has thin walled portions, a large lumen, an atraumatic end, a kink resistant construction and/or any combination thereof. Additionally, the access sheath of various embodiments of the present invention has an extensive range of lengths from about 5.5 centimeters or less all the way up to 100 centimeters or more and various lengths there between, e.g., about 13 and 45 centimeters. The access sheath of various embodiments of the present invention is also strong, stiff and yet flexible enough to be intricately guided through the body conduits, cavities or tissue.

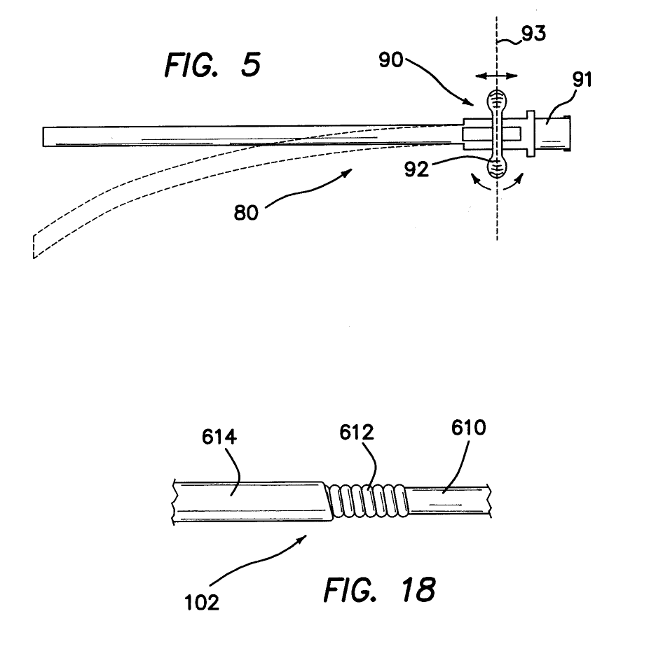

Referring now to FIG. 5, an embodiment of an actuator or actuation hand-piece 90 adapted to be in line with the access sheath 80 is shown. The proximal end of the actuator 90 includes a funnel-shaped entry 91 connected within the actuator to access a working channel, which forms a transition into the primary lumen of the access sheath. The entry 91 is also sized and arranged to receive surgical instruments such as a dilator 82.

A pull wire extending through a first secondary lumen of the access sheath 80 is attached to a first movable component, e.g., a threaded cylinder or ratcheted slider Another pull wire extending through a second secondary lumen of the access sheath is also attached to a second movable component. The first secondary lumen extends through the access sheath along a first side of the access sheath. The second secondary lumen also extends through the access sheath 80 along a second side of the access sheath. The first side of the access sheath opposes the second side of the access sheath.

A knob 92 surrounding the movable components is correspondingly threaded or otherwise arranged to engage the components, which allows a user with a twist or turn of the knob 92 in one direction, e.g., clockwise or proximally, to move one of the movable components linearly. For example, as the first movable component is moved proximally when the knob 92 is rotated clockwise or dragged proximally, the tensioning device connected to the first movable component also traverses towards the proximal end of the access sheath to impart a pulling force on the access sheath thereby deflecting the access sheath in a first direction.

The knob 92 is also allowed to move in the opposite direction moving the first movable component distally to straighten the access sheath. As the knob 92 continues to move in the opposite direction and past a zero point 93, the knob 92 disengages from the first movable component and engages the second movable component The tensioning device connected to the second movable component traverses proximally as the knob 92 traverses distally to impart a pulling force on the access sheath 80 thereby deflecting the access sheath in a different or opposing direction. The knob 92 moved in the opposite direction back towards the zero point 93 moves the second movable component distally to cause the tensioning device to loosen and thus allow the access sheath to straighten.

The various access devices and their construction described below may also be applicable to the steerable access sheath described above. For example, the remote actuation hand-pieces to be described below can be used instead of the ring shown in FIG. 1.

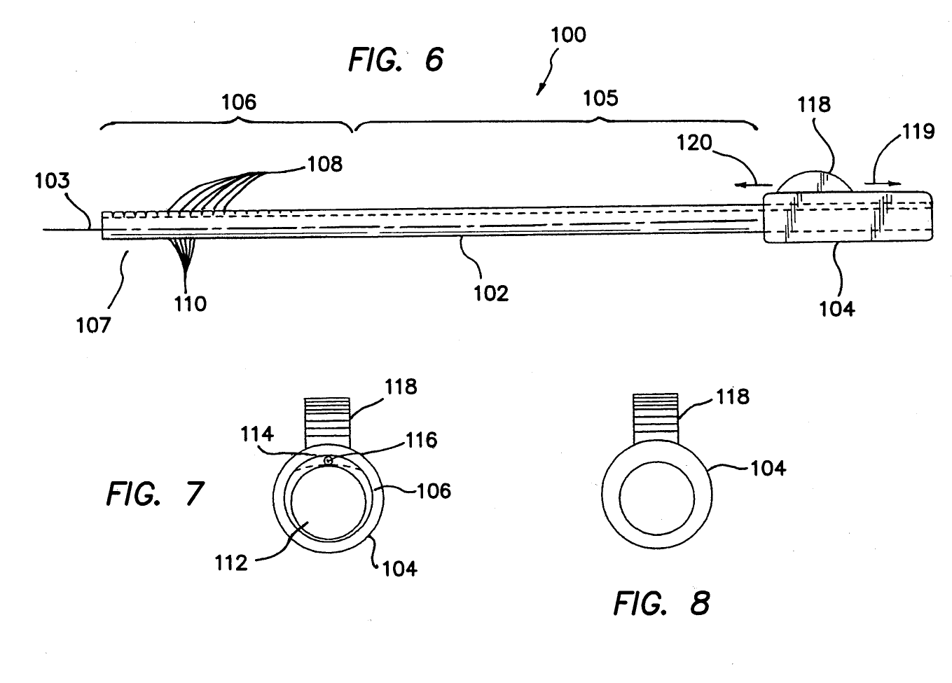

FIGS. 6-8 illustrate a surgical access device or steerable kink resistant access device 100 in accordance with the one embodiment of the present invention for use in, among other fields, cardiology, urology, radiology, electrophysiology and gastroenterology. Access device 100 comprises an access sheath 102 having a longitudinal axis 103 extending from a proximal end to a distal end, and a handle portion 104 operatively connected to the proximal end of the access sheath 102. The access sheath 102 includes an elongated body 105 and a steerable region or portion 106. It is appreciated that the steerable portion 106 may be formed anywhere along the access sheath 102. It is further appreciated that the steerable portion 106 and the elongated body 105 may have variable stiffness depending on the application of the access sheath 102. The access sheath 102 has an outside diameter sufficiently small so that it may be inserted into a body cavity or conduit. The access sheath 102 typically has two internal lumens, a primary lumen 112 and a secondary lumen 114, as illustrated in FIG. 7.

The primary lumen 112 is sized and configured as an access to a surgical site or the target of a surgical procedure. In particular, primary lumen 112 operates to advance diagnostic and therapeutic elements to the surgical site or target. The secondary lumen 114 is sized and configured to contain a tensioning device 116 such as a control or pull wire that, when acted upon, will deflect the steerable portion 106 of the access sheath 102. The tensioning device 116 extends through the secondary lumen 114 and is attached to the actuator or handle portion 104 at one end and to a distal portion 107 of the steerable portion 106 at the other end. The handle portion 104 may include a thumb-actuated knob 118 controlling the tensioning device 116. For example, the knob 118 may be drawn proximally in a direction 119 to provide tension to the tensioning device 116 or cause the tensioning device to tense or distally in a direction 120 to loosen tension or cause the tensioning device 116 to loosen.

It is appreciated that the actuator or actuation hand-piece of the invention may be remotely attached to the associated access sheath to control the tensioning and loosening of the tensioning device. In this case, the hand-piece may be connected to a flexible tubing or body, which is connected to the access sheath. By providing a remote access point or attachment, the thumbwheels of the hand-piece, for example, may be placed away from the surgical site so that they do not prevent or interfere with full insertion of the working length of the access sheath. It is further appreciated that the access sheath may comprise a plurality of pull wires attached to a plurality of thumbwheels of an actuation hand-piece to deflect the steerable portion of the sheath in different directions.

In one embodiment of the invention, the access sheath 102 comprises an extruded multi-lumen plastic tube. Alternatively, the access sheath 102 may be molded from a plastic or rubber-like material. Preferred materials include polyvinyl chloride, polyester, silicone elastomer, natural or synthetic rubber, polyurethane or the like. The materials may range in hardness from around 40 Shore A to 70 Shore D. These materials are generally flexible and durable. In another embodiment of the invention as illustrated in FIG. 18, a structure such as a spring can be molded into the tube of the sheath to facilitate kink resistance. More specifically, the access sheath 102 may be formed with an inner plastic body 610, surrounded by a metal spring coil 612, which is further covered by an outer body 614. This particular embodiment of access sheath 102 provides a high degree of kink resistance. The inner body 610 provides a smooth surface within the sheath, which facilitates passage of instrumentation. The spring coil 612 adds kink resistance to the sheath tube, while the outer body 614 provides a suitable covering for the coils of the spring 612.

In one aspect of steerability of the present invention, a tightly wound spring may be placed in the secondary lumen 114 of the access sheath 102 to facilitate movement of the tensioning device 116 inserted there through. The spring may be bonded or otherwise fixed to the secondary lumen 114. Among other features, the spring operates to isolate forces applied by the tensioning device 116, which is inserted through the spring and is attached to the distal portion 107 of the steerable portion 106. In particular, the spring adds stability and rigidity to the elongate body 105 when the tensioning device 116 is acted upon such that only the steerable portion 106 is bent or steered. Furthermore, the spring operates to direct the tension force applied on the device 116 to the steerable portion 106 so as to allow deflection of only the portion 106 and not the elongate body 105. That is, the tension force is isolated to the steerable portion 106, which may be formed anywhere along the access sheath 102 The spring may be coated with a lubricious material further facilitating movement of the tensioning device 116. The spring may line or cover the inner surface area of the entire secondary lumen 114 or just portions of the secondary lumen 114 to facilitate isolation of the tension force.

The spring may be constructed from a 0.005-inch diameter wire that is tightly wound forming a closed wound spring having a 0.02-inch outer diameter. The distal 0.5 to 2 inches of the spring may be stretched to an open wound state such that the windings have an approximately 0.02-inch gap between them. This stretched portion of the spring facilitates isolation of the tension force applied by the tensioning device 116. The spring may be coated, for example, in a plastic jacket and bonded to the secondary lumen 114 from the proximal end of the spring to the proximal end of the stretched portion. The stretched portion is then left free to move and/or compress in the plastic jacket. The distal end of the stretched portion may be anchored to the distal end of the access sheath 102 along with the tensioning device 116. The distal end of the plastic jacket may also be bonded to the distal end of the access sheath 102 along with the tensioning device 116 and the spring although these elements do not require a common bonding point or bonding method.

As discussed above, the proximal end of the access sheath 102 may be directly or remotely attached to handle portion 104 or actuator or hand-piece 900, which allows the operator to place tension on the tensioning device 116, such as a control or pull wire, while maintaining the position of the catheter. This tension causes the stretched portion of the 0.02-inch diameter spring to collapse and this, in turn, forces the sheath to bend in the region where the stretched portion of the spring is located. It is appreciated that the stretched portion may be formed anywhere along the catheter or surgical access device that may require bending, and is not limited to the distal end of the device. In addition, more than one deflection assembly of spring and tensioning device may be added to the access device to create deflection in different regions or planes. The amount of bending or deflection will in some way be proportional to the amount of force or tension placed on the tensioning device

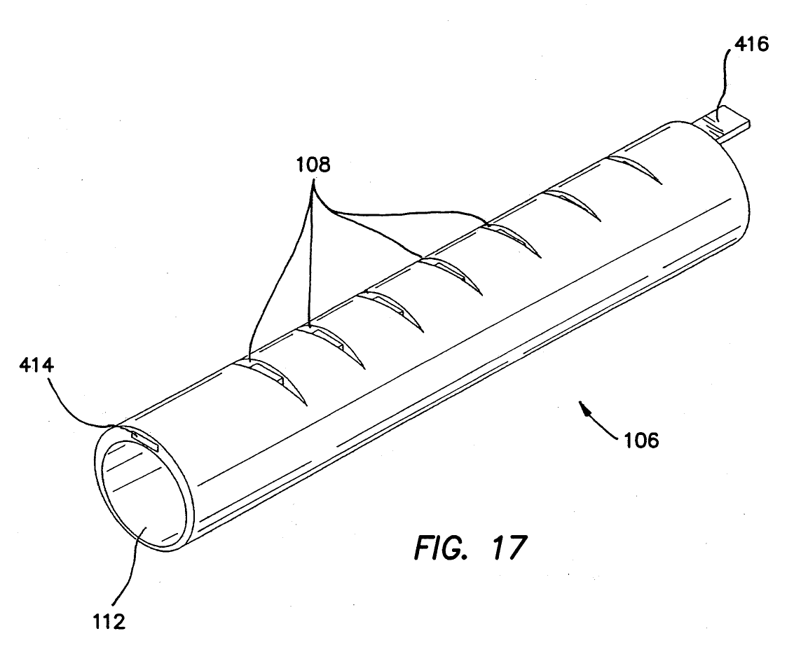

The tensioning device 116 is, in one embodiment, a control or pull wire made of Nitinol, a braided cable or any flexible strand or wire. In one embodiment, the control wire is inserted through the spring such that it runs through the secondary lumen 114 as illustrated in FIG. 10. The proximal end of the tensioning device 116, e.g., a control or pull wire, is connected to an actuator such as the knob 118 of the handle portion 104. The distal end of the control or pull wire, as previously described, is attached to the distal portion 107 of steerable portion 106. In another aspect of the invention as illustrated in FIG. 17, the tensioning device 416 may be a flattened or flat member extending through at least the steerable portion 106 of the access sheath 102

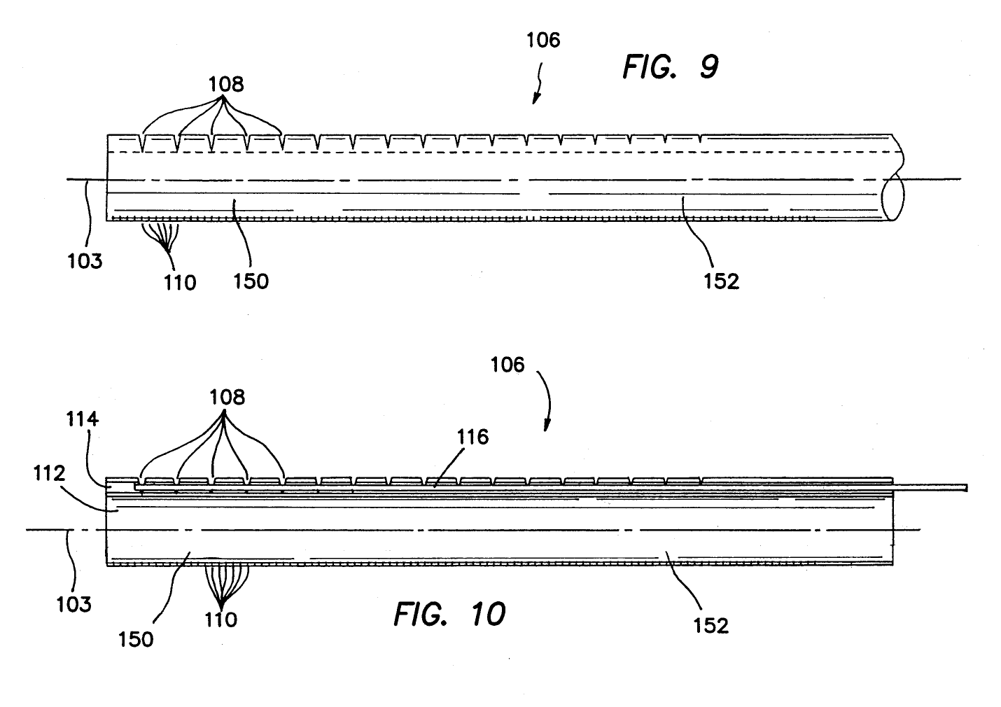

In another aspect of the present invention as illustrated in FIGS. 6 and 9-10, the steerable portion 106 includes a plurality of radially and longitudinally spaced notches 108 and slits 110 disposed on opposite sides of each other facilitating radial deflection of the distal portion 107 in a desired direction or angle. The notches 108 and slits 110 are cut into the access sheath 102 across the longitudinal axis 103. The degree of deflection may vary greatly based on many factors such as the number, size, direction, shape and spacing of the notches 108 and slits 110. The notches 108 are cut deeper and wider at a distal end 150 than they are at a proximal end 152 of steerable portion 106. The slits 110 comprise of very shallow cuts to provide a reduction in resistance to stretching as the steerable portion 106 is bent or deflected toward the notches 108.

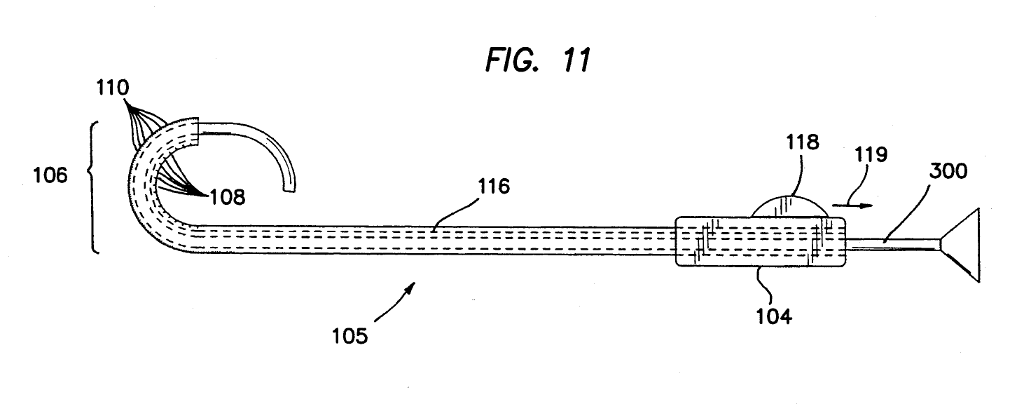

As discussed above, the notches 108 and slits 110 may be of any desired width, length, depth and shape. The number of notches 108 and slits 110 in the steerable portion 106 can be varied in accordance with the use and flexure requirements of the access sheath 102 However, in one embodiment, the slits 110 are narrower and shallower than the notches 108 to provide a “weak-side/strong-side” arrangement of the steerable portion 106 so as to allow the access sheath 102 to be predisposed to bending in the desired direction. That is, when the control wire of the tensioning device 116 is drawn proximally as illustrated in FIG. 11, the more flexible side of the steerable portion 106, i.e., the side with notches 108, will give first thereby bending in the direction of the notches Moreover, the distal end 150 of the steerable portion 106 with the deeper and wider notches 108 will bend first as the bending progressively moves toward the proximal end 152 having shallower and narrower notches. It is appreciated that the notches 108 may extend through the wall of the access sheath 102.

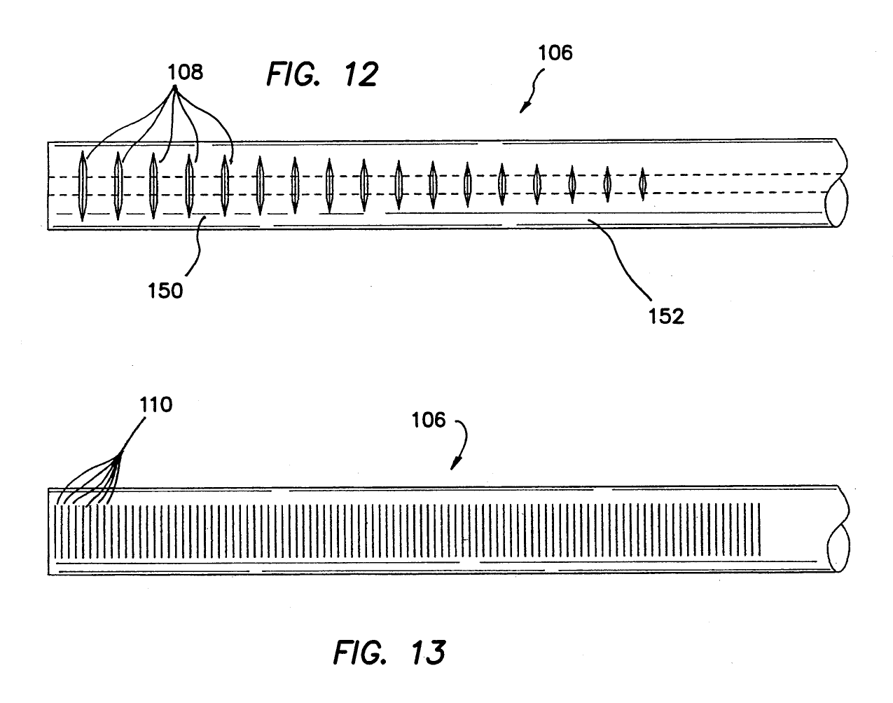

Referring now to FIGS. 12 and 13, the opposing series of notches 108 and slits 110 are further illustrated. The notches 108, as discussed above, provide a “weak-side” or preferred bend path as the notches 108 are closed when bent It can be seen that the notches 108 are wedge-shaped and have material removed from them. There is, therefore, sufficient room for the material adjacent to each notch to approximate, thereby shortening the length of the steerable portion 106 on the weak-side. In contrast, the slits 110 are shallow radial cuts made directly opposite the notches 108 with little or no material removed. The slits 110 provide the mechanical equivalent of increased plastic elasticity. That is, the slits 110 allow the material of the steerable portion 106 to stretch beyond the intrinsic properties of the material itself. As a result of this construction, the primary lumen 112 of the steerable portion 106 will not collapse when deformed or bent into a tight circular profile as can be seen in FIG. 11. In other words, the slits 110 will only open to provide an elongation of the “strong-side” and will not collapse to provide a shortening of the “strong-side”. The material on either side of the notches 108 and slits 110 maintains the general elongate dimension and forms a continuum of the access sheath 102.

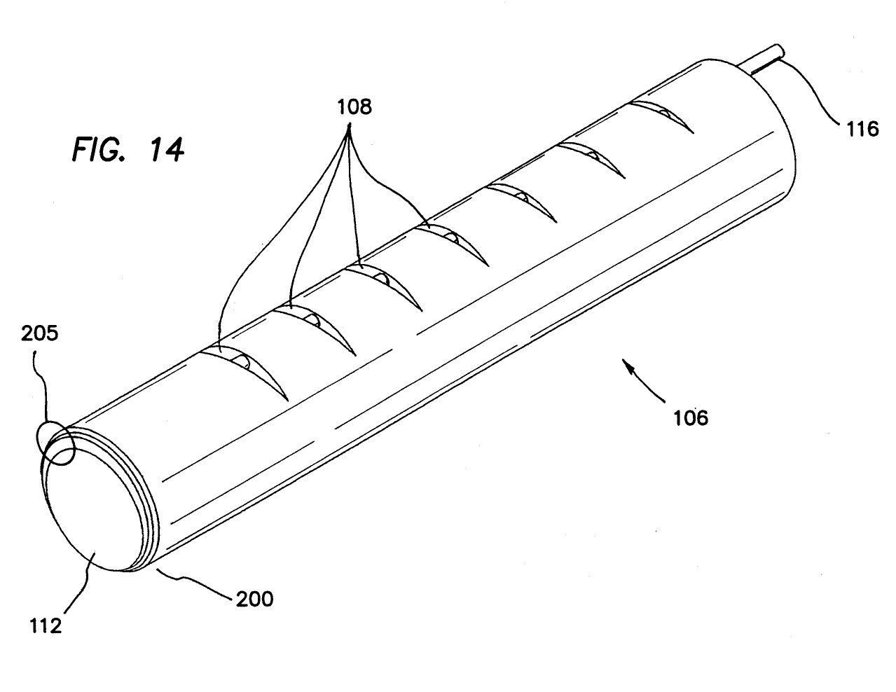

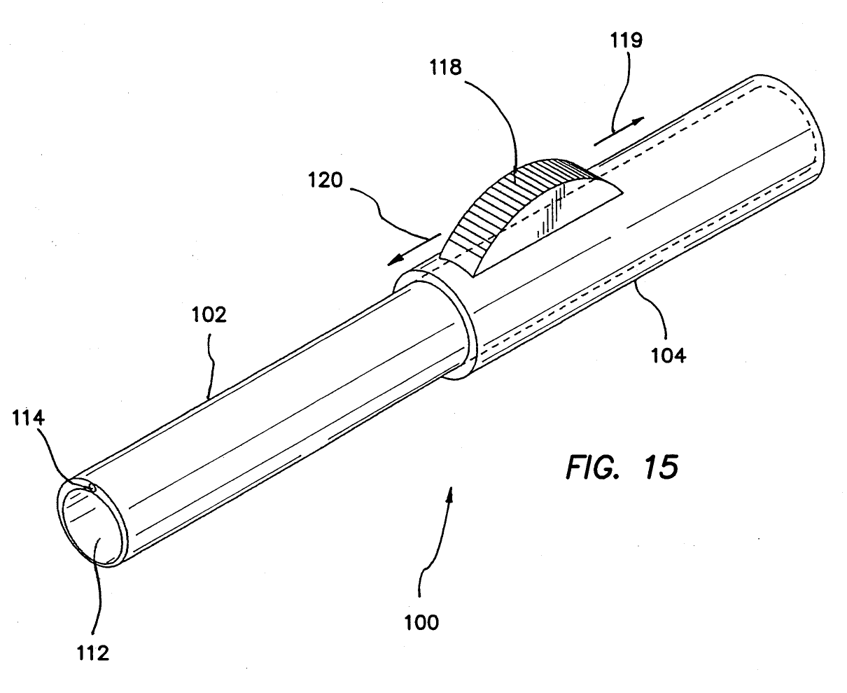

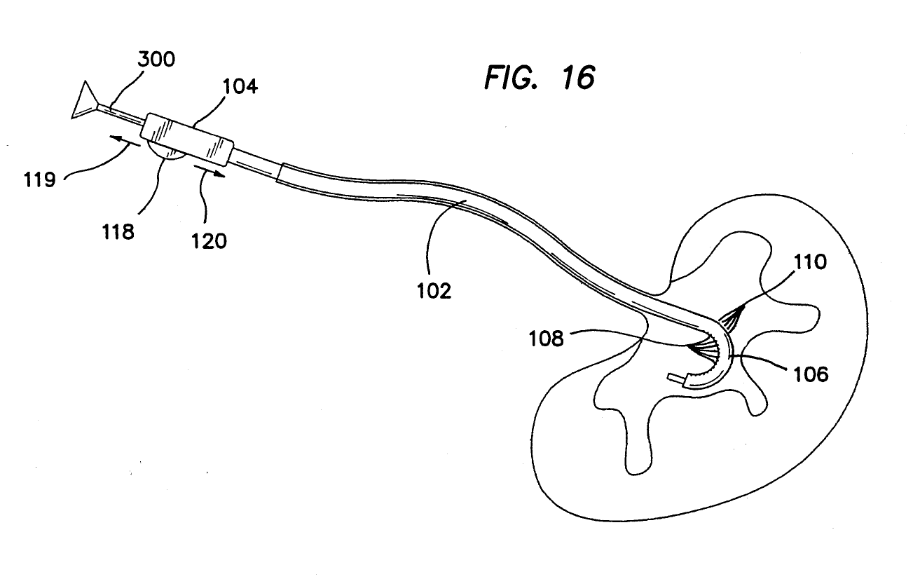

In another embodiment of the invention as illustrated in FIG. 14, the distal end 200 of the steerable portion 106 has a generally rounded off wall section 205 providing an atraumatic insertion tip. With the current construction of the access sheath having a steerable distal portion, less pushing force is required to advance the access sheath since it may be deflected around, under or over anomalies and irregularities in a body cavity or conduit rather than being forced through the tortuous paths. Surgical instruments such as an ureteroscope 300 may be directed through a steerable access sheath as illustrated in FIGS. 11 and 16. For instance, the steerable access sheath may be used to pass the ureteroscope 300 into the upper and lower poles of the renal calices as generally illustrated in FIG. 16. It is appreciated that flexible ureteroscopes and other flexible endoluminal scopes, including completely passive scopes, may be accurately positioned with the assistance of the steerable access sheaths of the present invention.

Fabricating the Access Sheath

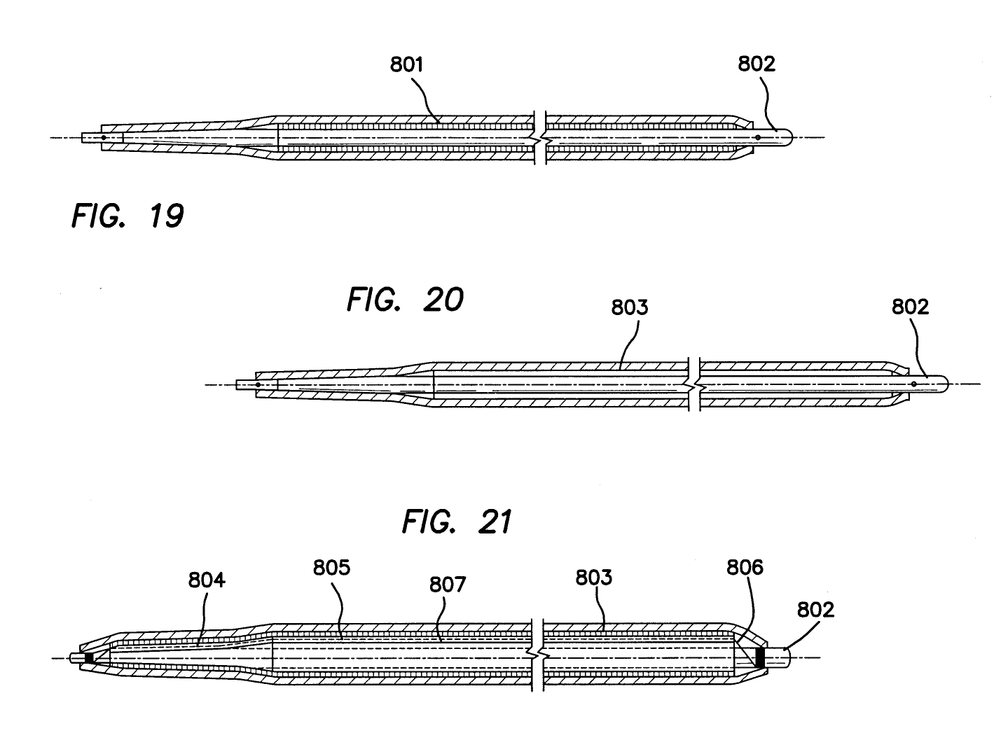

Referring now to FIGS. 19-22, embodiments of an access sheath in various stages of fabrication is shown. A wire 801 is wound around a support member or mandrel 802 in which the size and shape of mandrel generally defines the size and shape of primary lumen 112 of the access sheath 102. The mandrel, in one embodiment, is stainless steel and made of or is coated with a low friction material or surface, e.g., Teflon or various mold releases, allowing for the mandrel to be easily removed from the access sheath 102. The wire 801 is wound in an over counter fashion by using anchors or starting and stopping points substantially orthogonal of each other and thus winding the wire 801 in an oblique line along mandrel 802. As such, the wire 801 is wound such that the wire's tendency to unwind is counteracted. In one embodiment, prior to the addition of the wire 801, the mandrel 802 is coated with or inserted into a plastic or PVC material tube to allow instruments and the like to be smoothly inserted into the primary lumen without interference from the wire 801.

The wire 801, in one embodiment, is a plastic coated wire and particularly, a stainless steel co-extruded wire with an approximate diameter of 006 inches fused, coated or otherwise included with a plastic material to make the total diameter of the wire 801 to be about 0.012 inches. The mandrel 802 including wire 801 is placed into or inserted into a control tube. Air, in one embodiment, is supplied, e g., at 100 PSI, on the opposite end of insertion to assist insertion of the mandrel 802 by expanding the control tube. The control tube, in one embodiment, may be made of silicon or a material with a higher melting point than the plastic coating of wire 801. This assembly is then heated such that the plastic coating of wire 801 melts and adheres to itself to form a generally continuous tubular structure or major tube 803. The control tube is then removed.

A minor tube 804 is placed on or included with the major tube 803. The minor tube 804 is longer than the major tube 803 and thus extends substantially further along the mandrel 802 than the major tube 803. Extending within a portion of the minor tube 804 is a generally tubular structure or inner tube 805 that is about as long as the major tube 803. In one embodiment, the inner tube 805 is made of polyimide and the minor tube 804 is made of carbothane that when heated adheres to the inner tube 805, the major tube 803 and other portions of the access sheath, which are described below, that surrounds the outer periphery of the minor tube 804.

The inner tube 805 within the minor tube 804 is adapted to receive the support wire 806. The size and shape of the support wire 806 along with the inner tube 805 generally defines the size and shape of the secondary lumen 114 of the access sheath 102. In one embodiment, the support wire is a stainless steel wire with a diameter of about 0.12 inches. The support wire 806 is secured to a proximal end of the mandrel 802, threaded through the inner tube 805 and the minor tube 804 and secured to the distal end of the mandrel 802. In one embodiment, the support wire 806 secures the minor tube 804 to the major tube 803.

The minor tube 804 extends along the mandrel 802 substantially more than the inner tube 805 In other words, the length of the minor tube 804 is longer than the inner tube 805. The minor tube 804 is also more flexible than the inner tube 805. As such, the portion from the end point of the inner tube 805 and/or the major tube 803 to near the end point of the minor tube 804 eventually defines the steerable portion 106 of the access sheath 102. In one embodiment, the minor tube 804 is shorter and less flexible than the inner tube 805. Thus, in this embodiment, the portion from the end point of the minor tube 804 and/or the major tube 803 to near the end point of the inner tube 805 eventually defines the steerable portion 106 of the access sheath 102.

In one embodiment, the minor tube 804, inner tube 805 and the major tube 803 are placed into a final tube to enclose the minor tube 804 and inner tube 805 between the major tube 803 and the final tube. This assembly is placed into or inserted into a control tube such that the assembly adheres or bonds together and then the control tube is removed.

In one embodiment, the minor tube 804 or the inner tube 805, whichever extends further, is rigid, e.g., a stainless steel tube, to assist in the deflection of the steerable region 106. As such, the rigidity of the minor tube 804 or inner tube 804 prevents the non-steerable portion of the access sheath 102 from bowing. As such, the tube shifts the force caused by the tensioning device 116 to deflect the steerable region directly towards or at the steerable region 106. Also, a rigid secondary lumen formed by the rigid tube may assist in the protection of the tensioning device and instruments inserted or withdrawn from the primary lumen.

A wire 807 is wound around the minor tube 804, the inner tube 805 and the major tube 803. In one embodiment, where the final tube is utilized, the wire 807 is also wound around the final tube. In one embodiment, the wire 807 is similar in construction or composition as that of wire 801 and/or extends slightly beyond the distal end of the minor tube 804 or inner tube 805.

A support tip, in one embodiment, is placed on a distal end or slightly beyond the distal end of the wire 807 to assist in securing the wire 807 around the minor tube 804 or inner tube 805 and/or to provide an atraumatic tip. The support tip may be a 75 Shore D material. The mandrel 802 with rest of the assembly is inserted into a control tube. As previously mentioned, air, in one embodiment, is supplied on the opposite end of insertion to assist insertion of the mandrel 802 by expanding the control tube In one aspect, a support tube is used to temporarily encompass the control tube when the tube is pressurized in the event the tube breaks down. The control tube with the assembly is heated such that the plastic coating of wire 807 melts and adheres to itself to form a generally continuous tubular structure or tube 808. The control tube is then removed. In one embodiment, the control tube and assembly are heated at around 165 degrees plus or minus about five to ten degrees for about ten to fifteen minutes. As such, an access sheath 102 with a variable flexibility is created.

The support wire 806 is disconnected from the mandrel 802. For example, the support wire 806 on the distal end of the mandrel 802 is cut and then the mandrel 802 is withdrawn from the access sheath 803. At or near the tip of the access sheath, a tensioning device, e.g., a pull wire, is attached and threaded to the minor tube 804 and inner tube 805 out the proximal end of the access sheath 102 for securing to an actuator. As such, the access sheath is deflectable and controllable.

In one embodiment, the tensioning device is knotted or looped around an opening or cut in the access sheath, the support tip and/or between loops in the wire 807 and back through itself. A catch wire threaded through the inner tube 805 and the minor tube 804 hooks or otherwise attaches to the tensioning device. The catch wire is removed out the proximal end of the access sheath thereby threading the tensioning device through and out the proximal end of the access sheath 102. As it is appreciated the support wire 806 has a diameter sufficiently larger than the diameter of the tensioning device, the catch wire or loops and hooks of the catch wire to permit easy passage of these devices through the secondary lumen of the access sheath 102. A secondary support tip, in one embodiment, is placed on the distal end of the access sheath 102 to assist in securing the tensioning device to the access sheath and/or to provide an atraumatic tip.

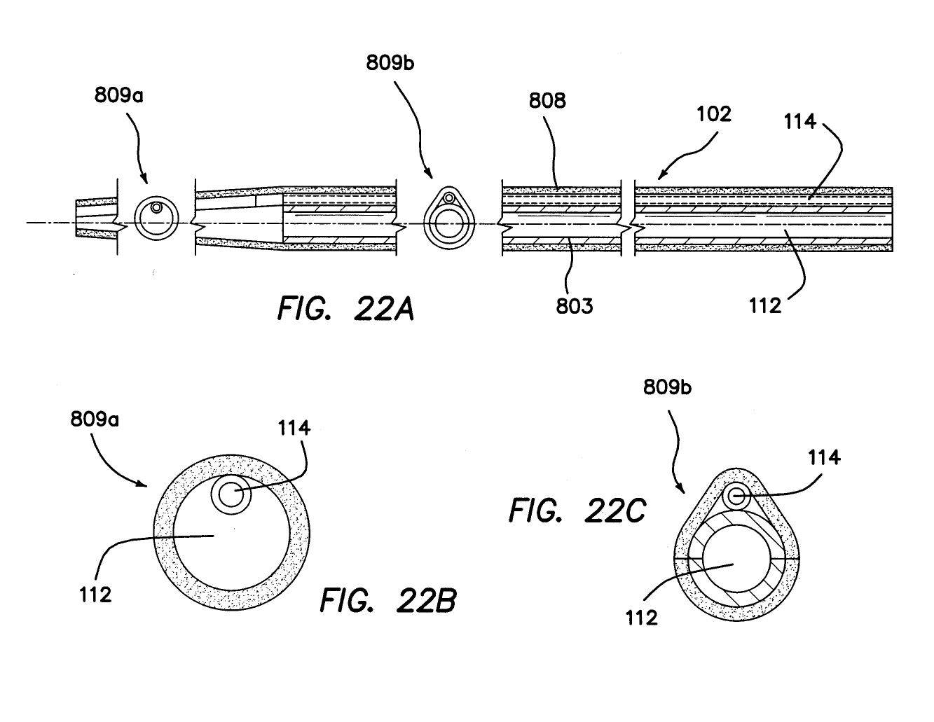

As shown in FIGS. 22A-C, the distal end 809a of the access sheath 102 is tapered and thus has a smaller diameter than the proximal end 809b of the access sheath 803. The primary lumen 112 and secondary lumen 114 diameters, however, remain substantially constant throughout the access sheath 102. Additionally, the tapering or reduced diameter of the access sheath is a result of the halting or non-extension of the inner tube 805 or minor tube 804, in one embodiment, and the major tube 803 along the length of the mandrel 802. As a result, the steerable portion 106 includes a reduced amount of materials and more flexible materials, and thus the steerable portion is easily deflected, bent, shaped or curved in response to the manipulation of the attached tensioning device while the other portion of the access sheath 102, including more material and less flexible material, remains substantially fixed, e.g., straight and substantially in the same plane, preventing any inadvertent or unintended movement of the access sheath.

Additionally, since the steerable region 106 of the access sheath 102 is reinforced by wire 807, the steerable region 106 is strengthen such that a flexible, pre-bendable or otherwise not actively controllable instrument may be controllably deflected dynamically as the steerable region 106 is controlled. Additionally, an actively deflectable surgical instrument may have a complicated construction providing components, e.g., optics or clamps, to perform its surgical function and components to perform the active deflection. Therefore, such instruments may be fragile or if broken may be expensive to replace or repair or still usable as a surgical instrument but not actively deflectable. As such, the strengthen steerable region 106 may replace the components or use of the components in such surgical instruments or induce an broken instrument to be controllably deflected thereby reducing replacement, repair and/or construction costs, reducing wear and tear of such instruments and increasing the life of such instruments. Also, the reinforced access sheath 102 through wire 807 and/or wire 801 allows the size and shape of the primary lumen to remain substantially constant throughout the access sheath 102, thereby reducing forces on instruments placed within the access sheath which may extend the life of these instruments.

The forces or stress accumulated along the access sheath that may cause kinks in the access sheath are also distributed along the access sheath due to the composite construction of the access sheath described above and are further counteracted by the wire coils, e.g., wire 807 and 803 Thus, kinks in the access sheath are reduced. The wire coils also allow the access sheath walls to be very thin without reducing durability or strength in the access sheath. Thus, the overall or outer diameter of the access sheath may be small, which may also reduce the incision or insertion point for the access sheath, without reducing the size or diameter of the primary lumen. As such, the access sheath of various embodiments of the present invention has thin walled portions, a large lumen, an atraumatic end, and a kink resistant construction and is strong, stiff and yet flexible enough to be intricately guided through the body cavity or tissue. In one embodiment, the wire coils are wound in a multifilar fashion with materials having alternating durometers.

Various other examples of processes that may be used to manufacture the access sheath 102 or portions of the access sheath 102 are described in U.S. patent application Ser. No. 101766,138 and U.S. Pat. No. 7,005,026, the disclosures of which are hereby incorporated by reference It is appreciated that these processes or portions of the processes may be varied or combined with the previously described process and vice versa. For example, various ring-shaped elements, such as, plastic rings, metallic rings, un-reinforced plastic rings and metal reinforced plastic rings, and the like may be utilized instead of or in addition to the wires 803 and/or 807. Additionally, a separate mandrel may be utilized to separately form or define the primary and secondary lumens and combined to make the access sheath.

Actuator or Handle for the Steerable Access Sheath

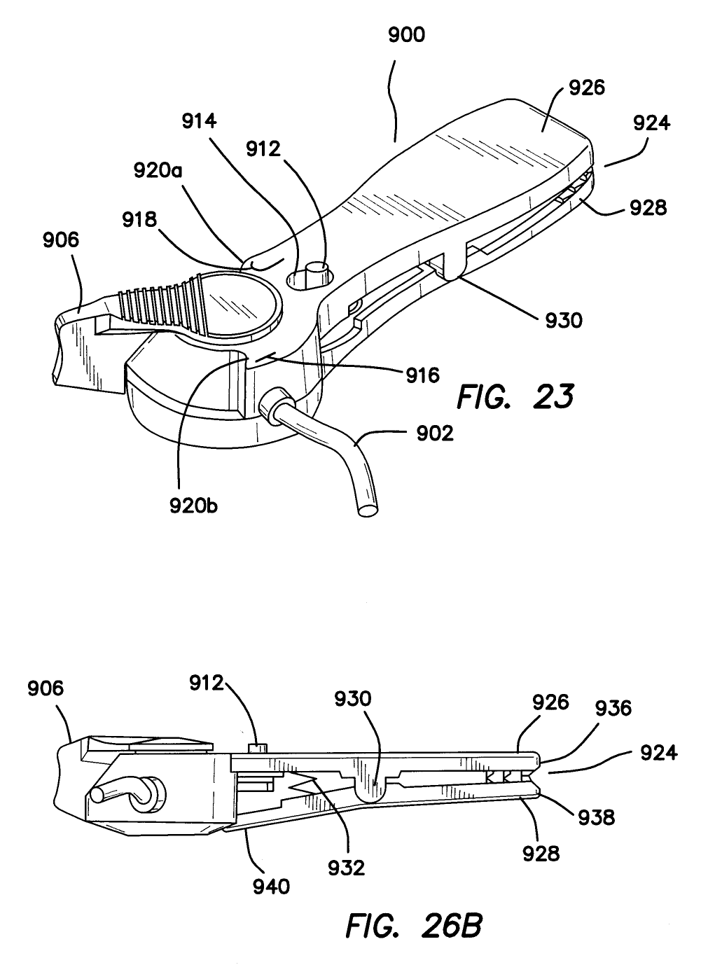

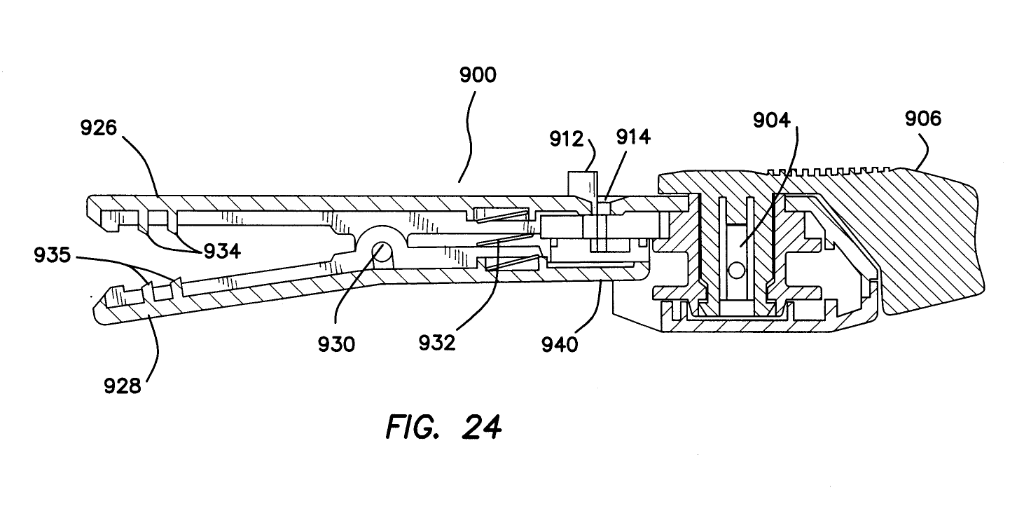

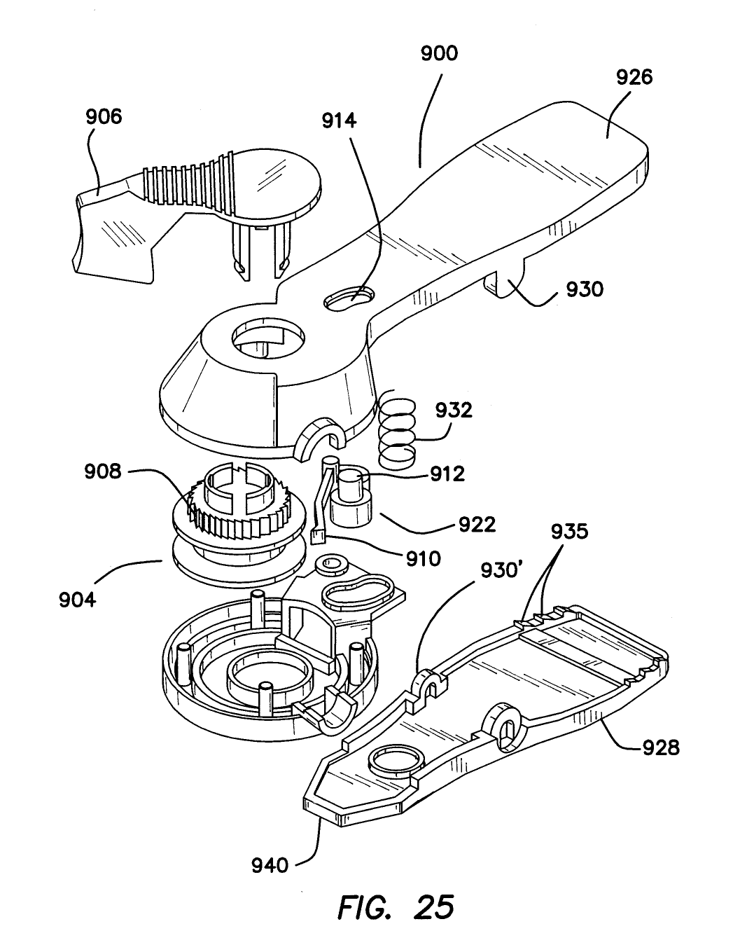

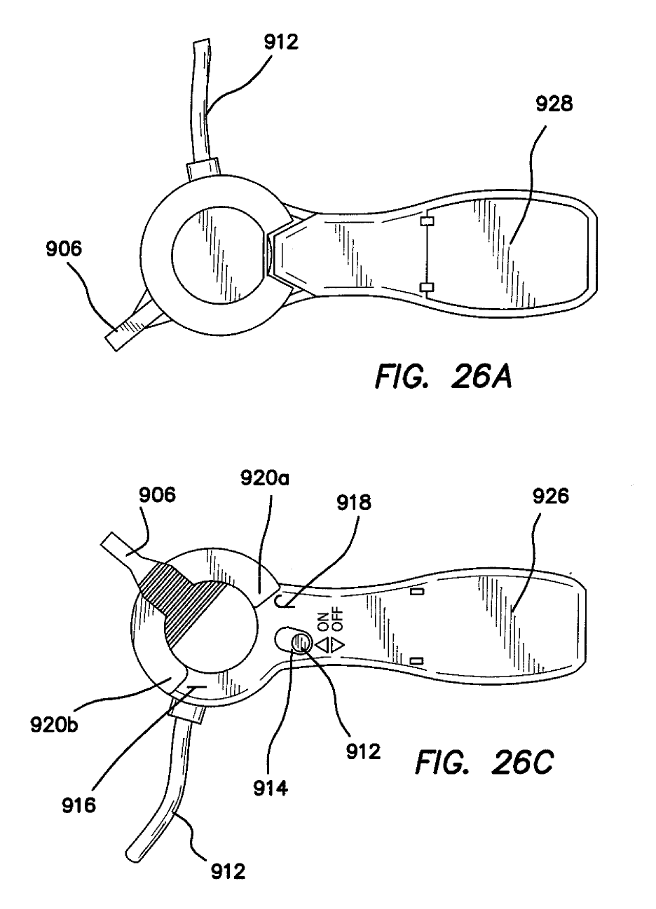

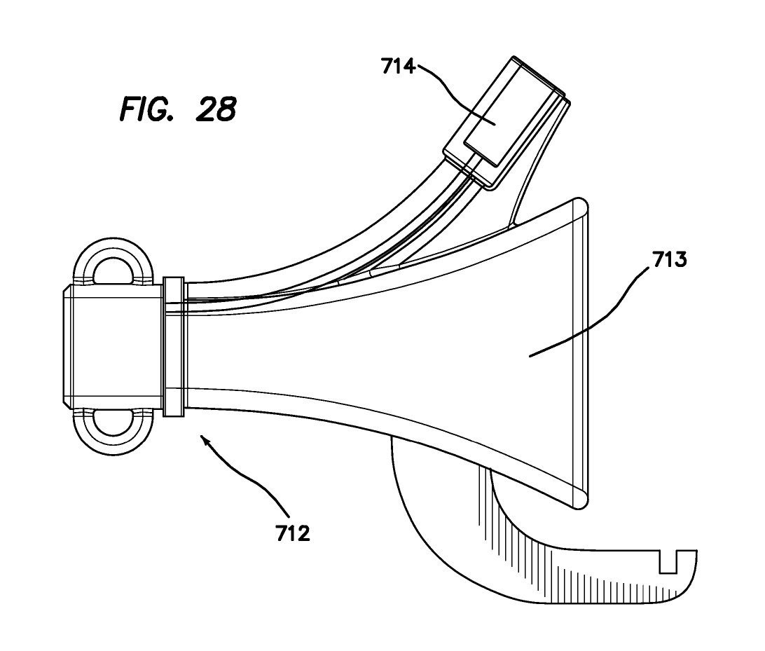

Referring now to FIGS. 23-28, an embodiment of an actuator or actuation hand-piece 900 of the present invention remotely attached to an access sheath is shown. The hand-piece 900, in one embodiment, is connected to a flexible body or conduit 902, which is connected to the access sheath 102 via a Y-connector 712. The Y-connector 712 includes a funnel-shaped entry portion 713 that is sized and arranged to guide instruments into the primary lumen 112 of the access sheath 102. The Y-connector 712 also includes a channel 714 for connecting to the flexible conduit 902. The tensioning device 116 extends through the secondary lumen 114, channel 714, and flexible conduit 902 and is attached to an axle 904 disposed within the hand-piece 900.

The axle 904 is also mechanically connected to a movable lever or thumb knob 906 extending from the hand-piece 900. The knob 906 allows a user to control the tensioning device 116 by rotating the axle 904. For example, when the knob is urged in one direction, e.g., rotating the axle clockwise, the tensioning device 116 is drawn proximally to wrap or wind around the axle 904 in the hand-piece 900, causing the steerable portion 106 of the sheath to deflect or bend. When the knob is urged in another direction, e.g., rotating the axle counterclockwise, the tensioning device is released to unwind and/or move distally from the axle, causing the steerable portion of the sheath to return it its original state, for example, straight or axially aligned with the non-steerable portion of the sheath.

The hand-piece design allows the operator to manipulate the knob, e.g. using a thumb, while holding the hand-piece in one hand, and is ergonomically shaped to accommodate all user hand sizes to provide comfort during use. As shown in FIG. 23, hard-stops 920 a,b are provided to limit the range of motion of the knob and to thus prevent over-winding of the tensioning device around the axle, avoiding potential breakage of the tensioning device.

Indicia, or directional indicators, provided on the hand-piece 900 relative to the knob 906 show the position of the steerable portion of the sheath. In FIG. 23, two such directional indicators are provided, at either end of the range of motion of the knob, one 916 showing the knob position corresponding to a steerable portion axially aligned with the nonsteerable portion of the sheath, the other 918 showing the knob position corresponding to the full range of deflection. As will be appreciated by the skilled artisan, indicia representing other intermediate states of deflection can also be included.

An activatable “on-off” mechanism is provided to permit incremental control of the steerable portion of the access sheath and to hold the tensioning device in place when the desired degree of sheath deflection is achieved. A plurality of teeth 908 radially disposed around the axle 904 or disposed on a ratchet wheel surrounding the axle operatively engages with a corresponding pawl or cantilever arm 910 of a loaded spring 922 mounted on the hand-piece 900 A button 912 on the loaded spring 922 extends from the hand-piece 900, through a slot or hole 914. The button may be moved between an “on” position and an “off” position.

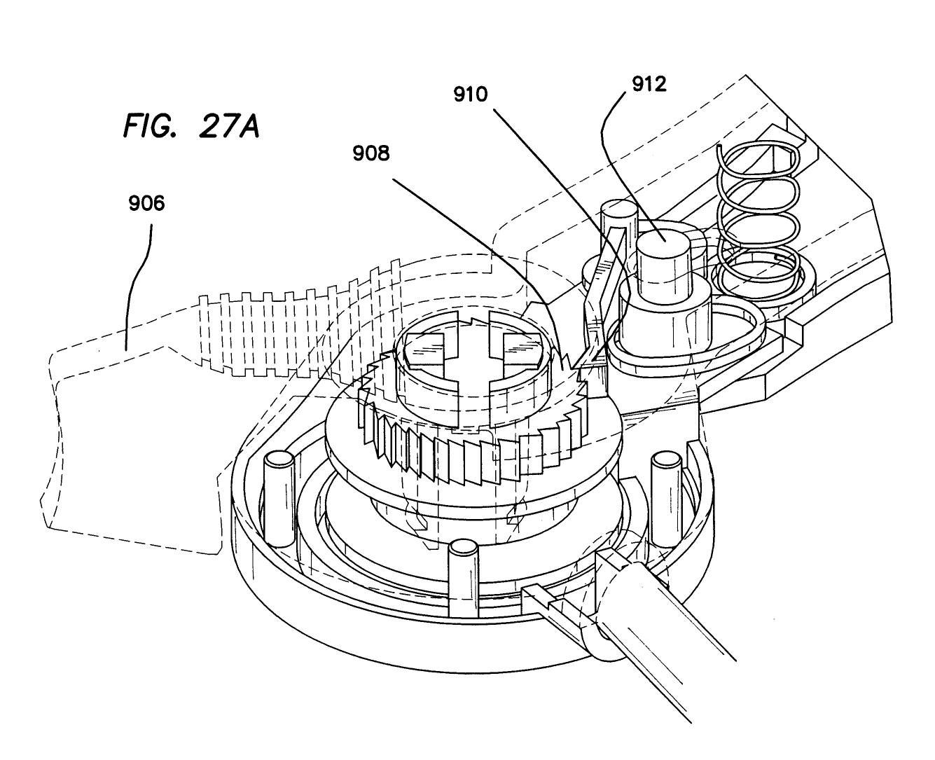

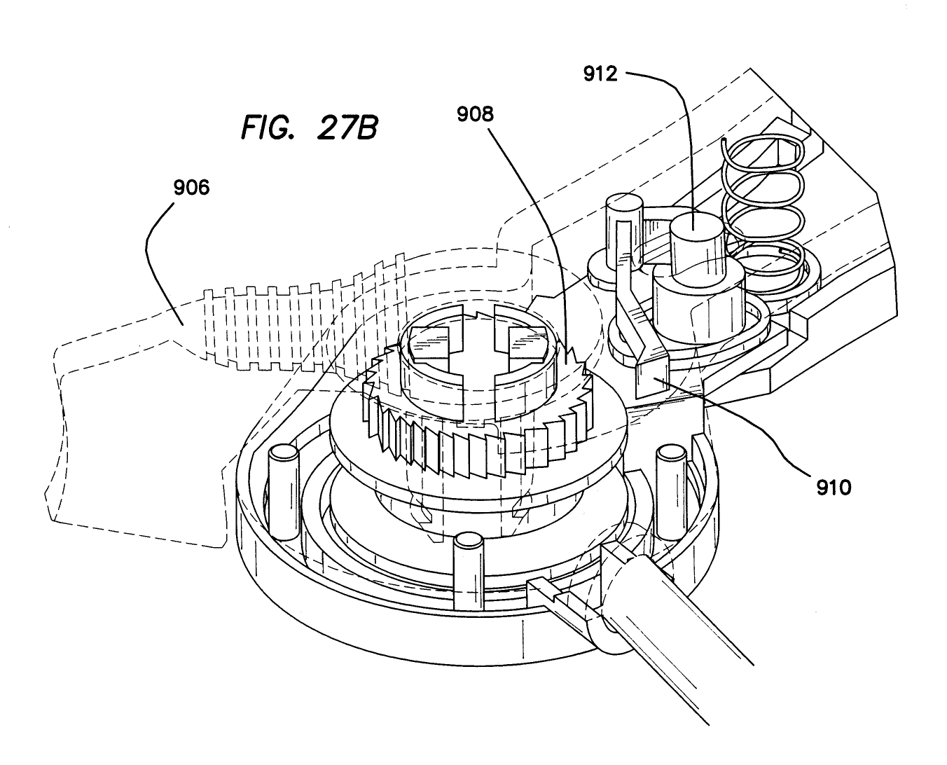

When the button 912 is urged in one direction, the “on” direction, the cantilever arm 910 engages with the teeth 908 to permit rotational movement of the axle 904, in one direction, e.g., a clockwise direction, while preventing rotational movement in the opposite direction. See FIG. 27(a). As such, as the axle 904 is turned clockwise, incremental control of the deflection of the steerable portion 106 of the access sheath is provided as the axle draws the tensioning device 116 proximally. As rotational movement in the opposite direction, e.g. counterclockwise, is prevented in this “on” position, the steerable portion of the sheath will remain in the deflected position until the axle is either rotated further to further deflect the steerable portion, or the button 912 in urged in the “off” direction.

When the button 912 is urged in another direction, the “off” direction, the cantilever arm 910 disengages the teeth 908 to permit free rotational movement of the axle 904. See FIG. 27(b). As a result, the tensioning device(s) unwind or move distally from the axle 904 whereby the steerable portion 106 of the access sheath 102 straightens.

In the embodiment shown in FIGS. 23-27, urging the button in a distal direction will engage the cantilever arm with the teeth to permit incremental axle rotation in one direction only (e.g., the “on” position); urging the button in a proximal direction will disengage the cantilever arm from the teeth to permit free axle rotation (e.g., the “off” position).

The embodiment shown in FIG. 23 also includes a clip 924, integrated into the hand-piece. The clip comprises an upper portion 926 and a lower portion 928, connected at a pivot point 930. A spring 932 is disposed between the upper and lower portions of the clip. Ridges 934, 935 on the proximal ends 936, 938 of the upper and lower portions provide gripping surfaces.

In the embodiment shown, the upper portion 926 of the clip is integral to the hand-piece 900, while the lower portion 928 of the clip rotates around the pivot point 930. The spring 932 is positioned distal to the pivot point, urging the proximal ends of the upper and lower portions of the clip together. When the distal end 940 of the lower portion 928 is urged toward the upper portion, compressing the spring 932, the proximal ends 936 and 938 move apart, allowing a material, such as a surgical drape, to be introduced between the ridges 934 and 935. When the distal end of the lower portion is released, the spring once more urges the proximal ends 936 and 938 together, “trapping” the introduced material between the ridges 934 and 935.

Use of the Steerable Access Sheath

In one embodiment of the present invention, various embodiments of access sheaths and actuators previously described, here now referred to as the access sheath, combined with an instrument or device used to stretch or enlarge an opening, e.g., a dilator, allows for gradual and atraumatic dilation of the ureter while being placed. Once the access sheath has been placed at a desired location, the dilator is removed and the access sheath is left in place. The access sheath allows for continued access to the desired area, for example, for the placement of an ureteroscope and other therapeutic instruments, while providing protection of the ureter. For instance, the access sheath may protect the ureter during the placement and removal of devices within the access sheath, during the removal of stone fragments or other tissue, and during the removal of a potentially cancerous biopsy specimen.

Additionally, with the access sheath being deflectable or steerable, an urologist may effectively and efficiently locate stones and stone fragments within the kidney. When a stone burden is found in one of the calyces of the kidney, especially in the lower pole portion of the kidney; it may be difficult for the urologist to continue to go back to the same calyx or location to remove the burden.

When there are many fragments within a calyx, many entries and exits may be performed to remove the burden Also, when a stone or stone fragment is removed, the instruments and tissue, e.g., the scope and stone basket (with the stone or stone fragment) are removed as a single unit. The scope is then passed back through the sheath and manipulated to find the same calyx in order to remove the remaining burden. However, with the access sheath 102, the access sheath can be left deflected in place looking at the same calyx or location, while the scope and stone basket are removed. As a result, the urologist's procedure time may be reduced, as the urologist may not have to manipulate the ureteroscope to look for the same calyx each time. The amount of time saved may be significant, especially if there is a large stone burden within the kidney. Additionally, the likelihood of doing damage to the kidney due to the additional manipulation that takes place every time the ureteroscope is placed back into the kidney may be reduced. Thus, with the access sheath, one can keep the sheath deflected towards a particular calyx and remove the stone burden without having to find the calyx each and every time a fragment is removed.

When the urologist manipulates an ureteroscope, the urologist may sometimes use the inside walls of the kidney to help deflect the ureteroscope to enter into a particular difficult locale. With the access sheath 102, instead of using the inside wall to help deflect the ureteroscope the access sheath may be used. Also, as previously mentioned, this will also help reduce the “wear and tear” on surgical instruments, such as ureteroscopes. The deflecting mechanism with the ureteroscope, if provided, can be damaged often and expensive repair The use of the access sheath may reduce the damage to the ureteroscope when it is used to help manipulate the ureteroscope to desired locations within the kidney.

The use of the access sheath 102 may also help a lesser-experienced urologist perform the same difficult procedure as their more experienced colleagues. In performing this procedure, the urologist may access the lower pole of the kidney in order to remove a stone burden. By performing this procedure in a retrograde fashion, one can reduce a patient's recovery time. If an urologist were neither skilled nor comfortable with using an ureteroscope in a retrograde fashion to remove a stone burden from a kidney's lower pole, the urologist would typically approach the stone burden in an antegrade fashion. This places a sheath percutaneously and thus may add additional recovery time for a patient as well as potentially increasing morbidity. But, with the access sheath 102 and an ureteroscope, an urologist may efficiently and effectively locate and remove a stone burden within the lower pole of a kidney. The access sheath can also be used in an antegrade fashion and will provide the same or similar features described above, however access in this manner may not be the preferred method.

Although the present invention has been described in certain specific aspects, many additional modifications and variations would be apparent to those skilled in the art. It is therefore to be understood that the present invention may be practiced otherwise than specifically described, including various changes in the size, shape and materials, without departing from the scope and spirit of the present invention. Thus, embodiments of the present invention should be considered in all respects as illustrative and not restrictive. Also, all the examples provided throughout the entire description should be considered in all respects as illustrative and not restrictive. The scope of the invention is, therefore, indicated by the following claims rather than by the foregoing description. All changes, modifications, and variations coming within the meaning and range of equivalency of the claims are to be considered within their scope.

We claim:

1. A handle for use with a steerable surgical device, comprising:
a housing;
an axle disposed within the housing;
a plurality of teeth radially disposed around the axle;
a movable knob connected to and outside the housing, the axle connected to the knob such that movement of the knob in a first direction causes the axle to rotate clockwise and movement of the knob in a second direction causes the axle to rotate counterclockwise;
at least one hard-stop on the housing, the hard-stop adapted to limit movement of the knob;
a loaded spring disposed within the housing, the loading spring having a cantilever arm adapted to engage the plurality of teeth around the axle and a movable button extending through an opening in the housing, such that movement of the button in a first direction causes the cantilever arm to engage the plurality of teeth and movement of the button in a second direction causes the cantilever arm to disengage from the plurality of teeth

2. The handle of claim 1, further comprising a flexible conduit attached to the housing.

3. The handle of claim 2, further comprising a wire attached to the axle and extending through the flexible conduit.

4. The handle of claim 1, further comprising a clip extending from the housing.

5. The handle of claim 1, further comprising a directional indicator on the housing.

6. A surgical access device comprising:
a tube having a substantially rigid portion and a substantially flexible portion extending from the substantially rigid portion, the tube including a primary lumen and a secondary lumen both extending through the tube, the secondary lumen having a pull wire extending through the secondary lumen and connected to the flexible portion of the elongated body;
a connector having a distal end connected to the tube, a proximal end including a funnel-shaped portion, and a channel disposed between the distal end and the proximal end, the pull wire extending through the connector from the distal end through the channel;
a flexible tubing connected to the channel through which the pull wire extends; and
a handle connected to the flexible tubing and including a housing, an axle disposed within the housing and connected to the pull wire, a plurality of teeth radially disposed around the axle, a movable knob connected to and outside the housing, the axle connected to the knob, and a loaded spring disposed within the housing, the loading spring having a cantilever arm adapted to engage the plurality of teeth around the axle and a movable button extending through an opening in the housing.

7. The surgical access device of claim 6, wherein the knob is adapted to control rotation of the axle

8. The surgical access device of claim 7, wherein movement of the knob in a first direction causes the axle to rotate clockwise and movement of the knob in a second direction causes the axle to rotate counterclockwise.

9. The surgical access device of claim 6, further comprising a hard-stop adapted to limit the movement of the knob in a first direction.

10. The surgical access device of claim 9, further comprising a second hard-stop adapted to limit the movement of the knob in a second direction.

11. The surgical access device of claim 6, wherein movement of the button in a first direction causes the cantilever arm to engage the plurality of teeth and movement of the button in a second direction causes the cantilever arm to disengage from the plurality of teeth.

12. The surgical access device of claim 11, wherein engagement of the cantilever arm with plurality of teeth prevents rotation of the axle in one direction while allowing incremental rotation of the axle in the opposite direction.

13. The surgical access device of claim 6, further comprising a clip extending m the housing.

14. The surgical access device of claim 6, further comprising a directional indicator.

15. The surgical access device of claim 6, further comprising means for incrementally controlling tension of the pull wire.

16. The surgical access device of claim 6, further comprising means for limiting the degree of sheath deflection.
